(19) United States
(12) Patent Application Publication
(10) Pub. No.: US 2015/0217061 A1
(43) Pub. Date: Aug. 6, 2015

(54) Needle Shield

(71) Applicant: Antares Pharma, Inc., Minneapolis, MN (US)

(72) Inventors: Peter L. Sadowski, Woodbury, MN (US); Michael Travanty, S.Minneapolis, MN (US); Kaushik J. Dave, Edison, NJ (US); Daniel Charles Stewart, Flemington, NJ (US); Patrick Madsen, Litchfield, MN (US); Julius Sund, Plymouth, MN (US)

(73) Assignee: ANTARES PHARMA, INC., Minneapolis, MN (US)

(21) Appl. No.: 14/422,538

(22) PCT Filed: Aug. 20, 2013
(86) PCT No.: PCT/US13/55647

§ 371 Date: Feb. 19, 2015

Related U.S. Application Data
(60) Provisional application No. 61/691,944, filed on Aug. 22, 2012.

(51) Int. Cl. A61M 5/32

(52) U.S. Cl. CPC A61M 5/3202; A61M 2005/312

(57) ABSTRACT

A needle shield comprises an elastomeric sheath having a closed distal end and an open proximal end. The distal end is configured to form a first seal with a distal tip of a needle in an initial position. The proximal end is configured to form a second seal with a syringe body in the initial position. The second seal is configured to release from the syringe body prior to the first seal releasing from the needle when the elastomeric sheath is moved distally from the syringe.

### CROSS-REFERENCE TO RELATED APPLICATIONS

This application claims the benefit of U.S. Provisional Patent Application No. 61/691,944 filed Aug. 22, 2012 entitled “Needle Shield”, which is incorporated by reference herein in its entirety.

### BACKGROUND OF THE INVENTION

The present invention generally relates to needle shields.

In order to seal a syringe, and more particularly to seal the needle projecting from the syringe to maintain sterility, a cap or needle shield is secured to the syringe and the distal end of the needle. Needle shields for syringes are well known and typically include a rigid plastic shell or cover with an elastic sheath therein.

Syringes containing drugs such as pre-filled syringes are a common alternative to vials and ampules as a primary container for medicaments to be injected. They offer advantages over their counterpart containers including simplified preparation for administration and in some applications, pre-measured dose. In pre-filled syringes with needles, needle shields constitute an important part of the barrier required to maintain the medicament and as such are directly or indirectly in contact with the medicament.

Needle shields are removably mounted to the needle end of a syringe to protect a user from pricks or sticks from the needle and to avoid exposure of the needle to contaminants. Needle shields also serve to seal the needle lumen that is in communication with the medicament chamber in the syringe. U.S. Pat. No. 4,986,818 discloses a typical needle shield that is removably mountable to a syringe to cover the needle tip and generally protect the needle and the needle tip from contamination because the needle is contained within the needle shield and the tip is located in the sheath in a storage configuration. The needle tip and needle are also stabilized by the sheath in the storage configuration to generally prevent damage to the needle and to limit user exposure to the needle tip.

### BRIEF SUMMARY OF THE INVENTION

In one embodiment, there is a needle shield comprising an elastomeric sheath having a closed distal end and an open proximal end, the distal end being configured to form a first seal with a distal tip of a needle in an initial position, the proximal end being configured to form a second seal with a syringe body in the initial position, the second seal being configured to release from the syringe body prior to the first seal being released from the needle when the elastomeric sheath is moved distally from the syringe.

In one embodiment, the elastomeric sheath is compressed by the needle to form a cavity housing the distal tip of the needle in the initial position, and wherein the elastomeric sheath expands to substantially fill the cavity upon removing the distal tip of the needle from the elastomeric sheath. In one embodiment, the elastomeric sheath is configured such that there is substantially no change in pressure within the elastomeric sheath proximate to the distal tip of the needle during removal of the distal tip of the needle from the elastomeric sheath. In one embodiment, an axial length of the first seal in the initial position is longer than an axial length of the second seal in the initial position.

In another embodiment, there is a syringe assembly comprising a syringe; a needle extending distally from the syringe and having a distal tip; and a needle shield having an elastomeric sheath having a closed distal end and an open proximal end, the distal end forming a first seal with the distal tip of the needle in an initial position, the proximal end forming a second seal with the syringe in the initial position, the second seal being configured to release from the syringe prior to the first seal being released from the needle when the elastomeric sheath is moved distally from the syringe.

### BRIEF DESCRIPTION OF THE SEVERAL VIEWS OF THE DRAWINGS

The foregoing summary, as well as the following detailed description of embodiments of the needle shield, will be better understood when read in conjunction with the appended drawings of exemplary embodiments. It should be understood, however, that the invention is not limited to the precise arrangements and instrumentalities shown.

In the drawings:

FIG. 1 is a side cross sectional view of a prior art needle shield shown mounted on a syringe;

FIG. 2 is a side view of a syringe and a needle shield in accordance with an exemplary embodiment of the present invention shown in an initial position;

FIG. 3A is a cross sectional view of the syringe and needle shield shown in FIG. 2 taken along a plane represented by line A-A;

FIG. 3B is a cross sectional view of the syringe and needle shield shown in FIG. 3A showing the needle shield being removed from the syringe;

FIG. 4 is a perspective view of a syringe in accordance with an exemplary embodiment of the present invention shown with the needle shield removed;

FIG. 5 is a side cross sectional view of the syringe shown in FIG. 4 shown with a needle shield in an initial position;

FIG. 6A is an end view of a needle shield in accordance with an exemplary embodiment of the present invention; and

FIG. 6B is a side cross sectional view of the needle shield shown in FIG. 6A.

### DETAILED DESCRIPTION OF THE INVENTION

The removal of a removably mounted needle shield from a syringe filled with medicament can result in the loss of medicament. This portion of medicament may remain inside the needle shield, as a drop hanging from the needle tip or simply fall to the ground. This loss can affect the dose of medicament available for injection and may cause exposure of the user, administrator or environment to undesirable medicament.

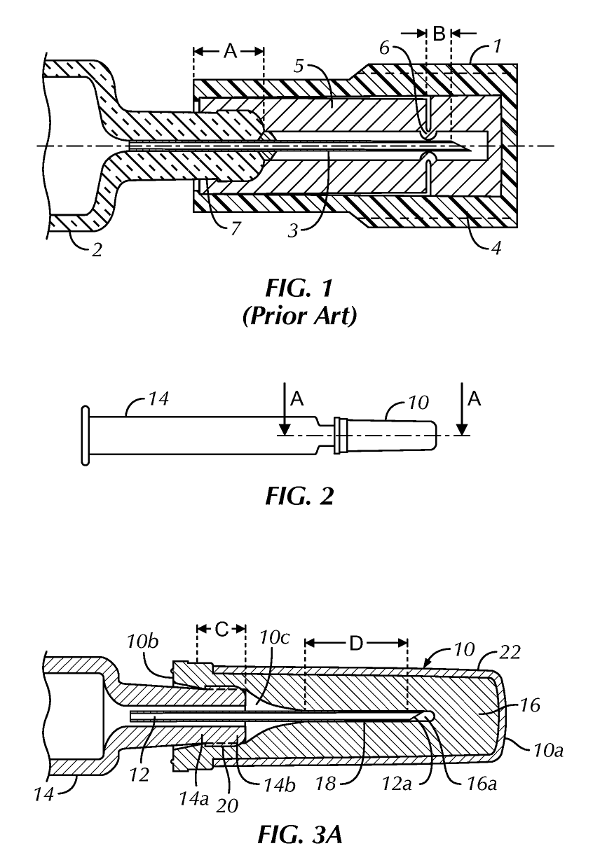

Referring to FIG. 1, a typical needle shield 1 covers a needle 3 extending from a syringe 2. Needle shield 1 may include a rigid casing 4 and an elastomeric sheath 5 therein. In the initial or stored position, elastomeric sheath 5 forms a first seal 6 with needle 3 and a second seal 7 with a distal end of syringe 2. An axial length B of first seal 6 in the initial position is less than an axial length A of second seal 7 in the initial position such that when removing needle shield 1 from syringe 2 releases first seal 6 from needle 3 prior to releasing second seal 7 from syringe 2. Because the cavity within elastomeric sheath 5 increases in volume as needle shield 1 is remove from syringe 2, second seal 7 prevents ambient air from entering the cavity and the pressure within the cavity drops. Moving first seal 6 along the tip of needle 3 increases the size of the cavity distal to the needle tip creating a pressure drop within the distal end of needle sheath 5. Also, releasing second seal 7 after releasing first seal 6 results in an additional pressure drop due to the cavity expansion between first seal 6 and second seal 7. Reducing the pressure within the elastomeric sheath 5 to a pressure below the pressure of the fluid within syringe 2 causes fluid within syringe 2 to be pulled through needle 3 and into needle shield 1 especially when needle shield 1 is removed slowly as the fluid has more time to move to the lower pressure. Pulling fluid from needle 3 and into needle shied 1 results in wasted fluid. The fluid may be expensive and even if the amount of wasted fluid is a few drops, a user may be frustrated that not all of the fluid is being used. Additionally, since the amount of fluid pulled through needle 3 depends upon the speed in which needle shield 1 is removed from syringe 2, the resulting dosage left in syringe 2 will vary which could be undesirable for the delivery of a specific dosage.

Referring to FIGS. 2-6B, wherein like reference numerals indicate like elements throughout, there is shown a needle shield, generally designated 10, in accordance with exemplary embodiments of the present invention.

Needle shield 10 and syringe 14, in some embodiments, are configured to reduce or eliminate pulling fluid from needle 12 upon removing needle shield 10 from syringe 14 regardless of the speed in which needle shield 10 is removed from the syringe 14. In some embodiments, minimizing the pressure drop caused by needle shield removal resulting in medicament being expelled from the medicament chamber of the syringe 14 is accomplished by configuring the seals between the needle shield 10 and syringe 14 and the needle shield 10 and a needle tip.

Referring to FIGS. 2-3B, in one embodiment, needle shield 10 has a generally elongated, cylindrical exterior shape. In other embodiments, needle shield 10 is rectangular, oval, triangular or any preferred shape in cross section. In one embodiment, needle shield 10 is open at the proximal end 10b and closed at the distal end 10a. In one embodiment, needle shield 10 tapers from proximal end 10b to distal end 10a.

In one embodiment, needle shield 10 includes a sheath 16. Needle shield 10 is configured to house a needle 12 extending distally from a syringe 14. In one embodiment, sheath 16 is configured to be radially displaced by needle 12. In one embodiment, sheath 16 is compressible. In one embodiment, sheath 16 is comprised of an elastomeric material. In one embodiment, sheath 16 is comprised of a thermoplastic elastomer (TPE) material. In one embodiment, sheath 16 includes a preformed slit, opening or cavity 16a configured to radially expand as needle 12 is forced distally into cavity 16a. In other embodiments, cavity 16a is substantially the same volume as the portion of needle 12 that it contains before and after receiving needle 12. In other embodiments, no preformed cavity 16a is provided and needle 12 forms cavity 16a by being forced through sheath 16 during insertion. In some embodiments, the cavity 16a increases in volume upon receiving needle 12 and then reduces in volume upon removal of needle 12.

In one embodiment, sheath 16 is at least partially surrounded by a housing or casing 22. In one embodiment, casing 22 may be provided to add rigidity to needle shield 10. In one embodiment, the rigidity of casing 22 is greater than the rigidity of sheath 16. In one embodiment, casing 22 is comprised of a polypropylene (PP) material. In one embodiment, sheath 16 is compression fit within casing 22. In other embodiments, sheath 16 is welded to casing 22 or secured to casing 22 using an adhesive. In one embodiment, sheath 16 and casing 22 are integral.

In one embodiment, sheath 16 is configured to form a first seal 18 with needle 12 in the initial position (FIGS. 2 and 3A) and a second seal 20 with syringe 14. In one embodiment, second seal 20 is shaped generally in the shape of distal end 14b of syringe 14. In one embodiment, distal end 14b is stepped or bulbous. In one embodiment, second seal 20 is configured to create a compression fit with syringe 14.

In one embodiment, first seal 18 is the last seal to be released from syringe 14 or needle 12 upon removing needle shield 10 from syringe 14 and needle 12. In one embodiment, first seal 18 has an axial length D in the initial position. In one embodiment, second seal 20 has an axial length C in the initial position. In one embodiment, there is a space between first seal 18 and second seal 20. In other embodiments, first and second seals 18, 20 form a continuous seal in the initial position. In one embodiment, axial length D of first seal 18 is longer than axial length C of second seal 20 such that when removing needle shield 10 distally from syringe 14, second seal 20 is released from syringe 14 before first seal 18 is released from needle 12. In one embodiment, once second seal 20 is released (see FIG. 3B) ambient air E enters the interior or cavity 10c of needle shield 10 while distal tip 12a of needle 12 remains sealed by at least a portion F of first seal 18.

Referring to FIG. 3B, in one embodiment, cavity 16a containing distal tip 12a of needle 12 in the initial position (FIGS. 2 and 3A) at least partially collapses as needle 12 is drawn proximally from sheath 16. In one embodiment, the maximum volume of cavity 16a during removal of sheath 16 from syringe 14 is equal or less than the volume of the needle 12. In one embodiment, the maximum empty volume of cavity 16a during removal of sheath 16 from syringe 14 is equal or less than the total volume of the cavity 16a in the initial position. In one embodiment, cavity 16a substantially collapses as needle 12 is drawn proximally from sheath 16. In one embodiment, the empty volume of cavity 16a remains generally constant during removal of sheath 16 from syringe 14.

In one embodiment, needle shield 10 and/or syringe 14 is configured such that the pressure within needle shield 10 upon removal from syringe 14 does not drop enough to pull any fluid within a prefilled syringe 14 through and out of distal tip 12a of needle 12. In one embodiment, sheath 16 and/or syringe 14 is configured such that less than 25 μl of fluid is pulled from needle 12 upon removal of needle shield 10 from syringe 14. In one embodiment, sheath 16 and/or syringe 14 is configured such that less than 20 μl of fluid is pulled from needle 12 upon removal of needle shield 10 from syringe 14. In one embodiment, sheath 16 and/or syringe 14 is configured such that less than 15 μl of fluid is pulled from needle 12 upon removal of needle shield 10 from syringe 14. In one embodiment, sheath 16 and/or syringe 14 is configured such that less than 10 μl of fluid is pulled from needle 12 upon removal of needle shield 10 from syringe 14. In one embodiment, sheath 16 and/or syringe 14 is configured such that less than 5 μl of fluid is pulled from needle 12 upon removal of needle shield 10 from syringe 14. In one embodiment, sheath 16 and/or syringe 14 is configured such that less than 1 μl of fluid is pulled from needle 12 upon removal of needle shield 10 from syringe 14.

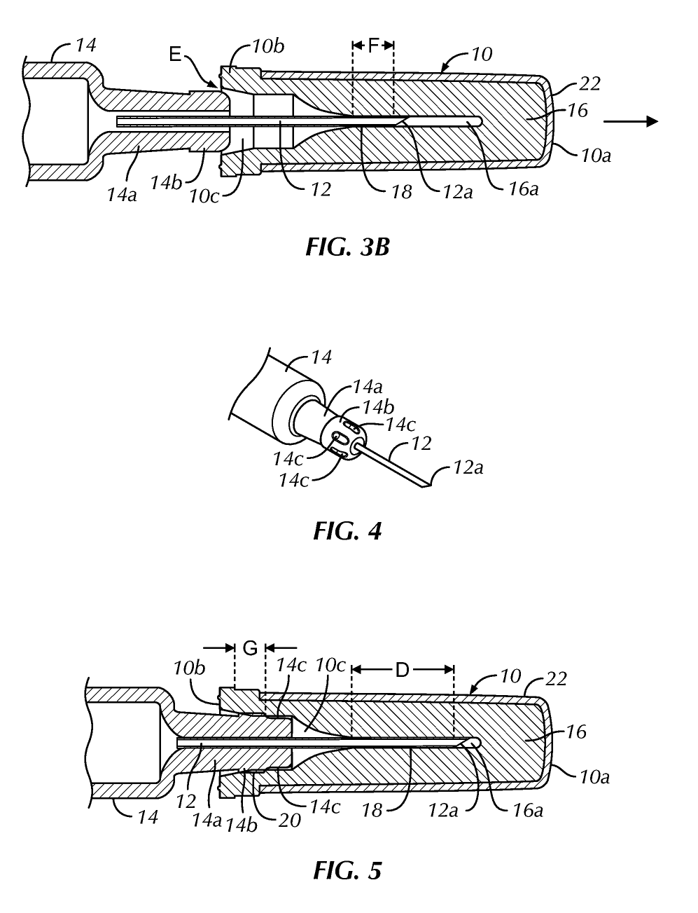

Referring to FIGS. 4 and 5, in one embodiment, sheath 16 is configured to form a first seal 18 with needle 12 in the initial position and a second seal 20 with syringe 14. In one embodiment, distal end 14b contains one or more radially projecting and axially extending grooves 14c. In one embodiment, distal end 14b contains six radially projecting and axially extending grooves 14c radially spaced equally around distal end 14b. In one embodiment, grooves 14c are configured to release second seal 20 before sheath 16 is removed from distal end 14b of syringe 14. In one embodiment, ambient air enters cavity 10c through grooves 14c before sheath 16 is released from syringe 14 and before first seal 18 is released. In one embodiment, grooves 14c allow for the axial length of the connection between sheath 16 and syringe 14 to be longer than the axial length G of second seal 20.

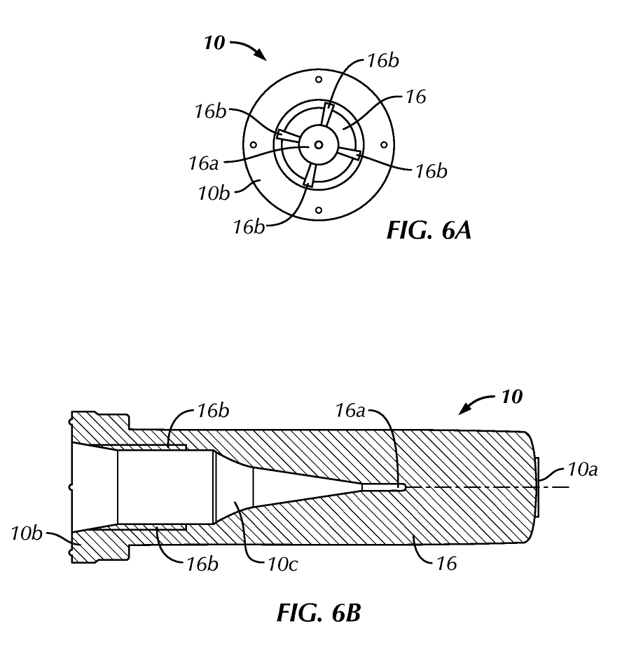

Referring to FIGS. 6A and 6B, in another embodiment, sheath 16 includes one or more radially projecting and axially extending grooves or vents 16b proximate proximal end 10b. In one embodiment, sheath 16 contains four radially projecting and axially extending grooves 16b radially spaced equally around sheath 16 proximal end 10b. In one embodiment, grooves 16b are configured to release a second seal 20 (see FIG. 5) before sheath 16 is removed from distal end 14b of syringe 14. In one embodiment, ambient air enters cavity 10c through grooves 16b before sheath 16 is released from syringe 14 and before first seal 18 is released. In one embodiment, providing one or more grooves 16b in sheath 16 allow for the axial length of the connection between sheath 16 and syringe 14 to be longer than the axial length G of second seal 20.

In another embodiment, sheath 16 includes one or more apertures configured to fluidly couple cavity 10c after second seal 20 has been released with cavity 16a distal to needle tip 12a such that cavity 16a is at ambient pressure prior to removing needle tip 12a from cavity 16a. In one embodiment, the aperture may be sealed by distal end 14b of syringe 14 such that the apertures fluidly couple cavity 10c with cavity 16a only after seal 20 has been released. In such an embodiment, second seal 20 is released prior to releasing first seal 18 but the aperture coupling cavity 10c with cavity 16a after second seal 20 is reduces any pressure drop in 16a that may otherwise be caused by removing needle 12. In one embodiment, either sheath 16a or syringe 14 include a projection to prevent needle shield 10 from being twisted relative to syringe 14 and aligning grooves 14c with the aperture.

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

I/we claim:

1. A needle shield comprising:
an elastomeric sheath having a closed distal end and an open proximal end, the distal end being configured to form a first seal with a distal tip of a needle in an initial position, the proximal end being configured to form a second seal with a syringe body in the initial position, the second seal being configured to release from the syringe body prior to the first seal being released from the needle when the elastomeric sheath is moved distally from the syringe.

2. The needle shield of claim 1, wherein the elastomeric sheath is compressed by the needle to form a cavity housing the distal tip of the needle in the initial position, and wherein the elastomeric sheath expands to substantially fill the cavity upon removing the distal tip of the needle from the elastomeric sheath.

3. The needle shield of claim 1, wherein the elastomeric sheath is configured such that there is substantially no change in pressure within the elastomeric sheath proximate to the distal tip of the needle during removal of the distal tip of the needle from the elastomeric sheath.

4. The needle shield of claim 1, wherein an axial length of the first seal in the initial position is longer than an axial length of the second seal in the initial position.

5. A syringe assembly comprising:
a syringe;
a needle extending distally from the syringe and having a distal tip; and
a needle shield having an elastomeric sheath having a closed distal end and an open proximal end, the distal end forming a first seal with the distal tip of the needle in an initial position, the proximal end forming a second seal with the syringe in the initial position, the second seal being configured to release from the syringe prior to the first seal being released from the needle when the elastomeric sheath is moved distally from the syringe.
